(19) United States
(12) Patent Application Publication
(10) Pub. No.: US 2026/0309590 A1
(43) Pub. Date: Oct. 8, 2026

(54) Sample probe

(71) Applicant: Teknologian tutkimuskeskus VTT Oy, Espoo (FI)

(72) Inventors: Vili Kellokumpu, Espoo (FI); Jussi Tenhunen, Espoo (FI); Hannu Vasama, Espoo (FI); Douglas Craig, Grappenhall, Warrington (GB); Dave McKeown, Grappenhall, Warrington (GB)

(21) Appl. No.: 19/482,797

(22) PCT Filed: Apr. 29, 2024
(86) PCT No.: PCT/FI2024/050200

§ 371 Date: Nov. 10, 2025

(30) Foreign Application Priority Data
May 10, 2023 (FI) 20235529

(51) Int. Cl. G01N 1/18; G01N 1/10; G01N 1/14; G01N 21/35; G01N 21/85

(52) U.S. Cl. CPC G01N 1/18; G01N 1/14; G01N 21/35; G01N 21/85; G01N 2001/1031

(57) ABSTRACT

According to an example aspect of the present invention, there is provided a probe device comprising a sample conveyor tube connectable with a source of pressurized gas, a probe head connected with the sample conveyor tube, the probe head comprising a sampling mechanism configured to obtain a sample of granular material surrounding the probe head, and the sampling mechanism being configured to provide the sample to the sample conveyor tube, and the probe device being configured to employ pressurized gas from the source of pressurized gas to propel the sample from the probe head via the sample conveyor tube.

### FIELD

The present disclosure relates to sampling granular material, such as, for example, granular polyethylene terephtalate, PET.

### BACKGROUND

Granular material is used in many different applications, such as injection moulding of plastics or thermoplastic resins, chemical synthesis processes with granular feed material and sampling of natural processes, such as sand, for example.

Depending on the application at hand, information characterizing the granular material may be useful or even necessary in ensuring successful operation of the application. For example, in chemical synthesis processes, variation in quality of feed material may result in spoilt batches, which may have to be discarded.

### SUMMARY

According to some aspects, there is provided the subject-matter of the independent claims. Some embodiments are defined in the dependent claims.

According to a first aspect of the present disclosure, there is provided a probe device comprising a sample conveyor tube connectable with a source of pressurized gas, a probe head connected with the sample conveyor tube, the probe head comprising a sampling mechanism configured to obtain a sample of granular material surrounding the probe head, and the sampling mechanism being configured to provide the sample to the sample conveyor tube, and the probe device being configured to employ pressurized gas from the source of pressurized gas to propel the sample from the probe head via the sample conveyor tube.

### BRIEF DESCRIPTION OF THE DRAWINGS

FIG. 1A illustrates an example system in accordance with at least some embodiments of the present invention;

FIG. 1B illustrates an example system in accordance with at least some embodiments of the present invention;

FIG. 2A illustrates an example sampling mechanism in accordance with at least some embodiments of the present disclosure;

FIG. 2B illustrates an example sampling mechanism in accordance with at least some embodiments of the present disclosure;

FIG. 2C illustrates an example sampling mechanism in accordance with at least some embodiments of the present disclosure;

FIG. 2D illustrates an example sampling mechanism in accordance with at least some embodiments of the present disclosure;

FIG. 3A illustrates an example sampling mechanism in accordance with at least some embodiments of the present disclosure, and

FIG. 3B illustrates an example sampling mechanism in accordance with at least some embodiments of the present disclosure.

### EMBODIMENTS

Disclosed herein are processes to provide sampling of granular material, such as polyethylene terephtalate, PET, polyamide, PA, polycarbonate, PC, or thermoplastic resin, to enable monitoring of moisture content, shape, size, and/or a ratio between recycled and non-recycled feed material, for example The herein disclosed sampling processes and devices involve using pressurised air to propel a sample, which has been obtained using a sampling mechanism, to an analytic device, such as a spectroscopic analytic device, for analysis. This enables the benefit that samples may be obtained from a part of the process which is close to a part where the granular material is used, enhancing the usefulness of the thus obtained information characterizing the granular material. Further, samples may be obtained from plural locations of the process, as will be described herein below.

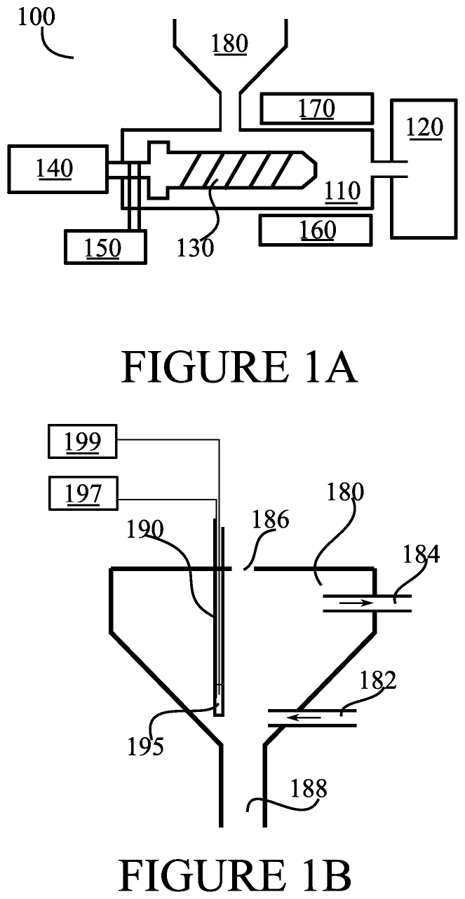

FIG. 1A illustrates an example system in accordance with at least some embodiments of the present invention. Injection moulding system 100 comprises a barrel 110 and a mould 120, into which PET is injected by piston 130. Mould 120 contains a shaped recess into which the PET material is injected, to obtain the desired object. Piston 130 may withdraw in part into injection cylinder 140 when retracted. In some systems, injection cylinder 140 is absent, in particular this is possible if the piston does not move much in the application in question. A motor 150 drives the piston, while heaters 160, 170 maintain the PET to be injected from barrel 110 to mould 120 sufficiently warm. Heat from heaters 160, 170 is arranged to be conducted to barrel 110.

PET granules are fed to barrel 110 from hopper 180. A hopper is discussed in more detail in connection with FIG. 1B, below. The granules may be a few millimetres, mm, in diameter. For example, the granules may be 2-4 mm in diameter. In practice, the granules are not necessarily of equal size, rather, their size will in general have a distribution which may depend on the lot, or lots, of PET being used. The hopper pre-warms the PET granules and dries them, since in order for a PET injection moulding process, or indeed another kind of process involving PET, to succeed, the PET material needs to be dry. Exactly how dry it needs to be depends on the specific application, but dryness requirements for granular PET exiting the hopper and entering barrel 110 may range from 10 parts per million, ppm, to 100 ppm water content. The more stringent dryness requirements, such as 10 ppm, are relevant when the PET is being processed to construct fabrics, known e.g. as polyester fabrics. Moisture content exceeding the dryness requirement is harmful to the manufacturing process.

PET granules used in various processes may be in part, or in whole, recycled polyester, rPET. Moisture content of rPET may vary from lot to lot, depending on the origin of the material. In practical applications, rPET may be mixed with PET which has not been recycled. As such, in order to meet the dryness requirement of the application in use, measuring the moisture content of PET granules is useful. Measuring the moisture content enables ensuring that the dryness requirement is met, while not over-drying the PET granules much beyond the dryness requirement, which would introduce delay into the process, as well as wasting energy in the form of heat used in drying. Further, excessively dry PET degrades the quality of products manufactured using the process. Thus it is optimal to ensure the moisture content is below the dryness requirement, but not excessively so. For example, the moisture content optimally be may be 75% to 80%, 80% to 85%, or 85% to 90% of the dryness requirement. As the dryness requirement relates to PET material used in the process, it is most useful to measure moisture content of the granular PET material as it enters barrel 110, rather than when it enters hopper 180. Hopper 180 may be furnished with a drying mechanism, as will be discussed in more detail in connection with FIG. 1B.

Sampling the PET granules from a place close to an opening where they leave hopper 180 also provides the benefit, that it can be verified that a ratio of rPET to PET is within a desired range. However, sampling the PET granules presents technical challenges. In detail, in particular near the interface of hopper 180 and barrel 110, the PET granules may collectively have a hard character, and furthermore individual granules tend to be electrostatic, causing them to stick to surfaces. The hard character is a result of high friction between PET granules, combined with the hardness of the granules themselves. The hard character is particularly prominent at the bottom part of hopper 180, from where samples are most preferably obtained for analysis.

FIG. 1B illustrates an example system in accordance with at least some embodiments of the present invention. Hopper 180 is here provided with hot air inlet 182 and cool air outlet 184, which together form a drying mechanism. Hot air inlet 182 is used to convey heated air into hopper 180, to dry PET granules in hopper 180 prior to their introduction into barrel 110 of FIG. 1A. Un-dried PET granules are fed into hopper 180 from opening 186 at the top. Dried PET granules are provided from hopper 180 to barrel 110 via opening 188 at the bottom. The air exiting hopper 180 via cool air outlet 184 is cool in the sense that it is cooler than air entering hopper 180 via hot air inlet 182. The heated air entering hopper 180 via hot air inlet 182 may be, for example, between 120 to 180 degrees Celsius, while the cool air exiting hopper 180 via cool air outlet 184 may be between 70 to 110 degrees Celsius, for example. The air is cooled by interaction with PET granules in hopper 180, this interaction including evaporation of water from the granules, and warming up the granules themselves. Since evaporation binds heat and warming the granules transfers heat from the heated air to the granules, the air is overall cooled down in the process.

The illustrated system further comprises a probe device, which comprises a sample conveyor tube 190 and a probe head 195. The probe device is arranged vertically in hopper 180, extending from the top of hopper 180 to a point over halfway from opening 186 at the top to opening 188 at the bottom. By vertical it is here meant, that the axis of sample conveyor tube is inclined by no more than 30 degrees. Sample conveyor tube 190 is coupled with a pressurized gas source 197. Pressurized gas source 197 may be a source of pressurized air or pressurized nitrogen, for example. Pressurized gas source 197 may comprise a container with pressurized gas inside, and/or a compressor configured to pressurize surrounding air, for example.

The system further comprises spectroscopic sensor device 199, which may comprise a near-infra-red, NIR, transmission spectroscopy device, for example. Spectroscopic sensor device 199 is arranged to receive samples of the granular material in hopper 180 from sample conveyor tube 190 and to determine the moisture content thereof, using spectroscopic methods, such as, for example, NIR transmission spectroscopy. An indication of the moisture content may be provided to users, and/or be used to automatically adjust a temperature and/or pressure at which hot air is provided to hopper 180 via hot air inlet 182. NIR transmission spectroscopy is preferable to NIR reflection spectroscopy, since reflection geometry is sensitive to surface moisture content and in a drying process, the surfaces of granules are dries first, leaving moisture inside the granule. When using NIR transmission spectroscopy, the sample is preferably sufficiently large, that NIR light does not shine, in places, through the sample without passing through at least part of at least one granule, as such non-interacted light creates a glare of NIR which disturbs the measurement, as the moisture content measured in practical applications of PET, for example, is in the ppm range. A NIR spectroscopic sensor device may comprises at least one of: a fourier transform infrared spectrometer, FTIR, a spectrograph and a filter wheel. FTIR is a fourier transform-based spectroscopic technique wherein an IR spectrum is obtained, such that spectral data is obtained over a wide spectral range. This yields a wider wavelength range than dispersive spectrometers, for example. A spectrograph uses a device, such as a prism or grating, to differentiate wavelengths in light to be analysed, with one or more photodetectors to obtain spectral information. A filter wheel may be used to use several filters in succession to obtain spectral information. The filters may be notch filters, for example, which each admit only a narrow band of light, to enable sampling relevant parts of the NIR spectrum to obtain enough spectral information.

In some embodiments, spectroscopic sensor device 199 is configure to vibrate a sample holder before compressing the sample in the sample holder for transmission NIR spectroscopy for the moisture content measurement, to obtain a well distributed sample for transmission measurement lacking with minimized NIR glare.

The probe device further comprises a probe head 195, which is arranged to obtain samples of the granular material in hopper 180 and to provide these samples to sample conveyor tube 190. The probe head has a sampling mechanism to capture the samples and provide them to sample conveyor tube 190. The samples are propelled, using the pressurized gas, to spectroscopic sensor device 199 where their moisture content is determined.

A benefit of the vertically arranged sample conveyor tube 190, is that openings in the hopper lower housing and horizontal tubes in hopper 180 are minimized in number. This is useful, since openings in the hopper 180 lower housing are weak points, and horizontal tubes in the hopper, especially near the lower opening 188, tend to disturb the flow of material in the hopper. This does not occur as much when the sample conveyor tube is vertical, as in FIG. 1B.

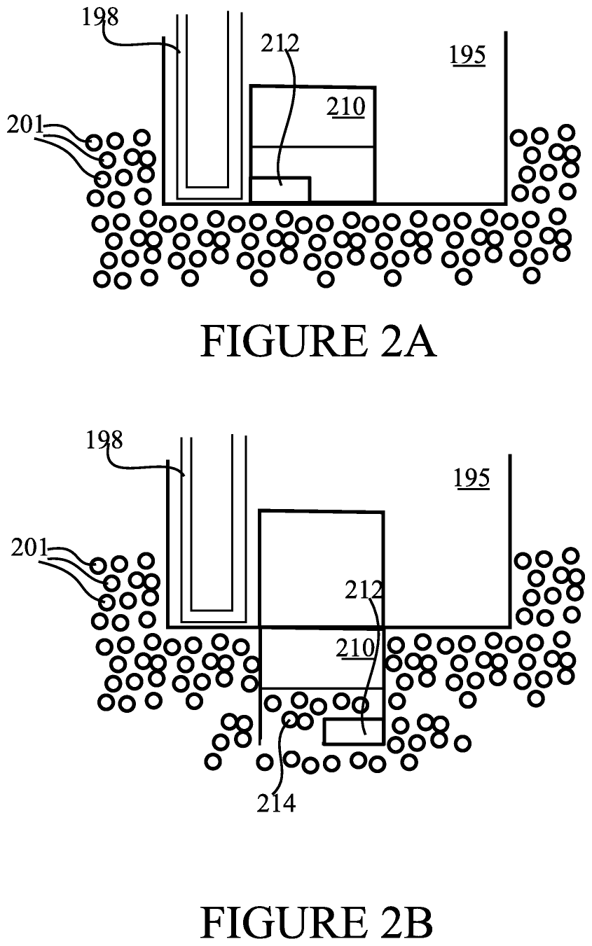

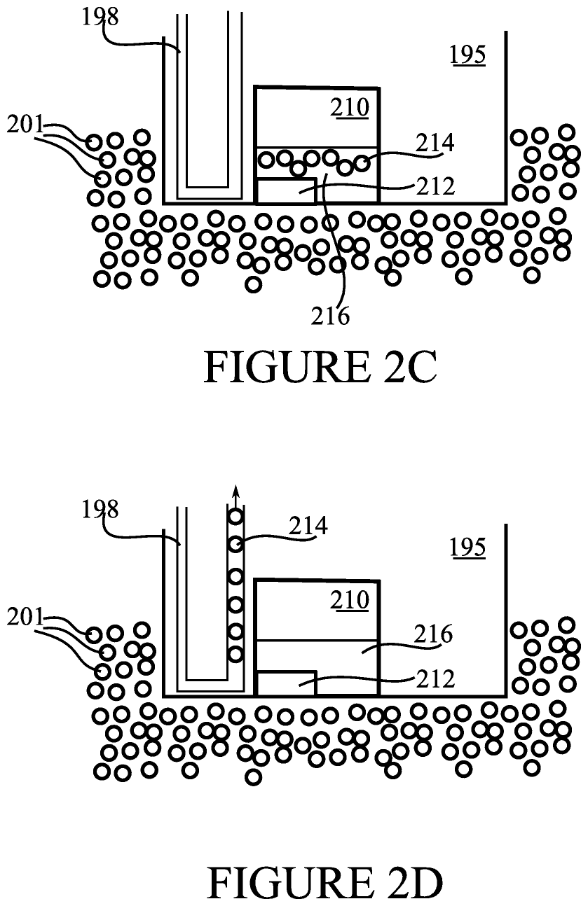

FIG. 2A illustrates an example sampling mechanism in accordance with at least some embodiments of the present disclosure. A lower end of probe head 195 is illustrated, surrounded by PET granules 201. Gas tube 198 is connected with pressurized gas source 197, which is outside the view of FIG. 2A. Sampling mechanism 210 is comprised in probe head 195, and in sampling mechanism 210 is located enclosing member 212. PET granules 201 around probe head 195 may be warm and tightly packed together, and exhibit significant static electrical charges. As illustrated in FIG. 1B, the probe head 195 is closer to the lower opening 188 than the upper opening 186 of hopper 180.

FIG. 2B illustrates an example sampling mechanism in accordance with at least some embodiments of the present disclosure. The device is the same one as in FIG. 2A, but in a different stage of operation. In detail, sampling mechanism 210 has been extended into the mass of PET granules 201, in the process creating a recess in the granular PET material 201 surrounding the probe head. Enclosing member 212 has further been moved to an open position, allowing some granules 201 to enter into a recess in sampling mechanism 210 and thus form sample 214. Sample 214 consists of the PET granules 214 which enter into a recess of sampling mechanism 210. The action of enclosing member 212 is schematically illustrated in FIGS. 2A and 2B, in practice the shape and action of the enclosing member may be implemented in several different ways. In particular, enclosing member 212 may be curved in shape and move along a curved rail. In general, enclosing member 212 is able to enclose, by physical movement of enclosing member 212, sample 214 in the recess in sampling mechanism 210.

FIG. 2C illustrates an example sampling mechanism in accordance with at least some embodiments of the present disclosure. The device is the same one as in FIG. 2A and FIG. 2B, in a different stage of operation. In detail, following the stage of FIG. 2B, enclosing member 212 has been moved to a closed position and sampling mechanism 210 has been retracted back inside probe head 195. Moving enclosing member 212 to the closed position encloses off the sample 214 from the granular material 201 surrounding the probe head 195. The sample 214 is thus in the recess 216 of the sampling mechanism 210. The PET granules 210 once more encompass probe head as in the situation of FIG. 2A, however now the sampling mechanism 210 comprises therein PET granules 214 of the sample, obtained by mechanically sampling the PET granules 201 surrounding probe head 195.

FIG. 2D illustrates an example sampling mechanism in accordance with at least some embodiments of the present disclosure. The device is the same one as in FIGS. 2A-2C, in a further stage of operation in which granules of the sample 214 have been moved to gas tube 198, and are being propelled by the pressurized gas from pressurized gas source 197 toward spectroscopic sensor device 199. How the granules 214 of the sample are transferred to gas tube 198 from recess 216 of sampling mechanism 210 may be approached in several ways, for example, the gas tube 198 may be arranged to allow the gas to traverse the recess 216 in sampling mechanism 210 wherein the sample 214 is when the sampling mechanism is retracted into probe head 195. Alternatively, an ejector effect may be triggered using the pressurized gas to such the granules 214 of the sample from the recess 216 of sampling mechanism 210.

The sampling mechanism of FIGS. 2A-2D is effective in sampling warm, dry PET granules in a lower hopper environment without unduly disturbing the functioning of hopper 180. In one or more stages of the sampling process illustrated in FIGS. 2A-2D, the sampling mechanism may be caused to vibrate, using a suitable source of vibration installed in probe head 195, to dislodge PET granules stuck in the mechanism. For example, static electricity may cause PET granules to get stuck preventing movement of enclosing member 212 to the closed position. As another example, the PET granules may get stuck in a mechanism transferring the granules from recess 216 to gas tune 198. Vibration may dislodge the granules and enable the mechanisms to complete their action.

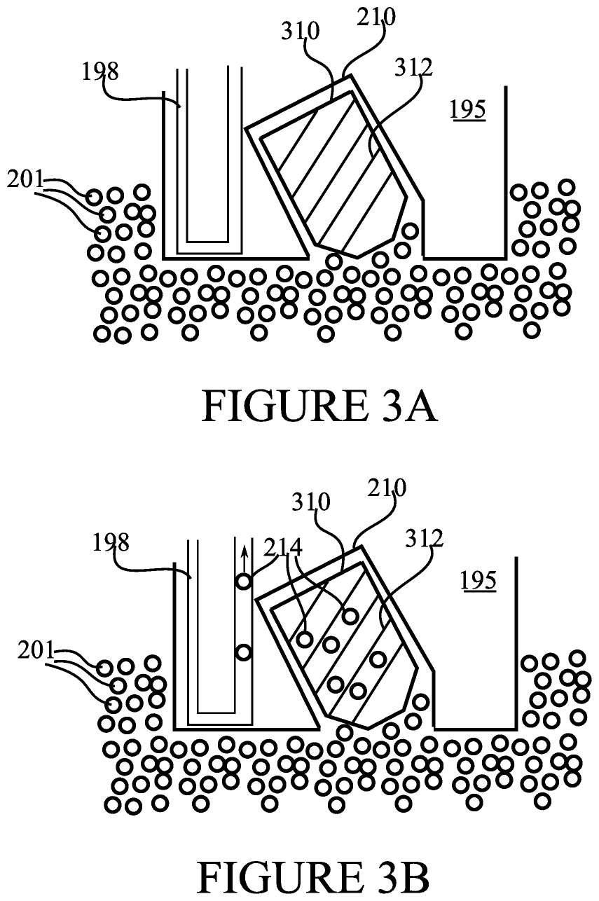

FIG. 3A illustrates an example sampling mechanism in accordance with at least some embodiments of the present disclosure. like numbering denotes like structure as in FIGS. 2A-2D. The sampling mechanism of FIGS. 3A and 3B is based on a screw part 310, which is in the stage illustrated in FIG. 3A immobile in probe head 195. Screw part 310 has a male thread 312 thereon, creating a meandering recess threading along an outer surface of screw part 310, the walls of the recess being formed of male thread 312.

FIG. 3B illustrates an example sampling mechanism in accordance with at least some embodiments of the present disclosure. The device is the same one as in FIG. 3A, in a further stage of operation.

In detail, screw part 310 has been caused to rotate about its longitudinal axis, in the process trapping some granules 201 to the meandering recess 316 threading along the outer surface of screw part 310. The granules so trapped are conveyed, by the rotation of screw part 310, into sampling mechanism 210. The granules so trapped and conveyed form sample 214. The rotation of screw part causes the sample 214 granules to be enclosed by the meandering recess and a wall of sampling mechanism 210. The sample 214 is thus enclosed in probe head 195.

Granules of sample 214 are here, as was the case in the embodiment of FIGS. 2A-2D, conveyed to gas tube 198 where they are propelled by the pressurized gas to spectroscopic sensor device 199 for analysis of their moisture content, and, optionally, the ratio of rPET to non-recycled PET. There are several ways to convey the granules of sample 214 from recess 316 to gas tube 198. For example, a side of sampling mechanism 210 may have a hole through which the granules may be sucked to gas tube 198 via a corresponding hole in a side of gas tube 198. Alternatively, for example, gas tube 198 may be arranged to pick up granules arriving at an end of recess 316.

As was the case in the embodiment of FIGS. 2A-2D, also the screw part 310, sampling mechanism 210 and/or gas tube 198 may be enabled to vibrate to dislodge granules which have become stuck due to random variation in motion, or static electricity. A motor arrangement configured to rotate screw part 310 may also be powerful enough to break granules 201 which are stuck. In terms of spectroscopically measuring moisture content or an rPET/PET ratio, it does not matter if some granules of sample 214 are broken. The rPET/PET ratio may be measured, alternatively to using spectroscopy, by using an image sensor with software configured to differentiate between rPET and PET granules in the material based on their visual appearance. For example, a machine vision application may be run on a processor, application-specific integrated circuit, ASIC, a field-programmable gate array, FPGA, or therein on one or more processing core of the processor, ASIC or FPGA.

In some embodiments, sample conveyor tube 190 is fitted with more than one sampling mechanism 210, to obtain samples from different depths of hopper 180. Some of the sampling mechanisms 210 may be of a type illustrated in FIGS. 2A-2D, and some of a type illustrated in FIGS. 3A-3B. In particular, the sampling mechanism based on screw part 310 may be more applicable deeper in hopper 180, while the sampling mechanism based on mobile enclosing member 212 may be suitable in use in lesser depths of hopper 180. While the sampling mechanisms of the figures attached hereto extend, at least in part, downward into PET granules 201, they may equally be arranged to extend horizontally into PET granules 201. Horizontal operation is particularly useful in case the sampling mechanism is not installed at the end of sample conveyor tube 190, at the probe head.

Whereas described herein primarily in terms of PET granules, the herein disclosed sampling mechanism is also applicable to other granules, such as thermoplastic resin granules, for example.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the preceding description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in sampling granular materials.

### ACRONYMS LIST

ASIC application-specific integrated circuit
FPGA field-programmable gate array
FTIR fourier transform infrared spectrometer
NIR near infra-red (780-2500 nanometer, nm, wavelength)
PET polyethylene terephtalate
ppm parts per million
rPET recycled polyethylene terephtalate

### REFERENCE SIGNS LIST

| | |
|---|---|
| 100 | injection moulding system |
| 110 | barrel |
| 120 | mould |
| 130 | piston |
| 140 | injection cylinder |
| 150 | motor |
| 160, 170 | heaters |
| 180 | hopper |
| 182 | hot air inlet |
| 184 | cool air outlet |
| 186, 188 | opening |
| 190 | sample conveyor tube |
| 195 | probe head |
| 197 | pressurized gas source |
| 198 | gas tube |
| 199 | spectroscopic sensor device |
| 201 | PET granules |
| 210 | sampling mechanism |
| 212 | enclosing member |
| 214 | sample |
| 216 | recess |
| 310 | screw part |
| 312 | male thread |
| 316 | recess |

1. A sampling device comprising a probe device wherein:
the probe device comprises a sample conveyor tube connectable with a source of pressurized gas;
the probe device comprises a probe head connected with the sample conveyor tube, the probe head comprising a sampling mechanism configured to obtain a sample of granular material surrounding the probe head; and
the sampling mechanism being configured to provide the sample to the sample conveyor tube, and the probe device being configured to employ pressurized gas from the source of pressurized gas to propel the sample from the probe head via the sample conveyor tube,
the sampling device further comprising a near infra-red, NIR, spectroscopic sensor device into which the sampling device is configured to convey the sample from the sample conveyor tube, and by the sampling device being configured to determine a moisture content of the sample using NIR spectroscopy of the sample in the sensor device, the sample being granular polyethylene terephtalate, PET, and in that the sampling device is arranged to operate with the sample conveyor tube and probe head inserted vertically into a hopper of a PET injection moulding apparatus.

2. The probe device according to claim 1, wherein the sampling mechanism is arranged to perform a two-phase operation to obtain the sample, a first phase of the two phases comprising creating, by mechanical movement of at least a part of the sampling mechanism, a recess in the granular material surrounding the probe head and a second phase of the two phases comprising enclosing the sample.

3. The probe device according to claim 2, wherein the sampling mechanism comprises a recess into which the sample is configured to be enclosed in the second phase by closing off the sample from the granular material surrounding the probe head by movement of an enclosing member comprised in the sampling mechanism.

4. The probe device according to claim 3, wherein the sampling mechanism is configured to vibrate to dislodge granules stuck to the sampling mechanism.

5. The probe device according to claim 1, wherein the sampling mechanism comprises a screw part which the probe device is configured to rotate to transport the sample into the probe head and enclose it in the probe head.

6. The probe device according to claim 5, wherein the screw part is configured to rotate in an oscillatory manner to dislodge granules stuck to the screw part.

7. (canceled)

8. (canceled)

9. The sampling device according to claim 18, further configured to perform the determining of the moisture content of the sample using NIR transmission spectroscopy of the sample in the sensor device.

10. The sampling device according to claim 17, wherein the NIR spectroscopic sensor device comprises at least one of: fourier transform infrared spectrometer, a spectrograph or a filter wheel.

11. The sampling device according to claim 18, configured to at least one of:
provide an indication of the determined moisture content to a user, or use the determined moisture content to automatically adjust a temperature and/or pressure of a PET drying process.

12. (canceled)

13. The sampling device according to claim 1, wherein the sample device further comprises an image sensor and a processing core, the processing core configured to determine, based on image data from the image sensor, shape and size of granulates and/or a ratio of recycled PET to unrecycled PET in the granular material surrounding the probe head.

14. A PET injection moulding apparatus comprising the sampling device according to claim 9, with the sample conveyor tube and probe head inserted vertically into the hopper.

15. The PET injection moulding apparatus according to claim 14, further comprising at least one second sampling mechanism connected with the sample conveyor tube.
